SEQUENCE LISTING (14 sequences)

SEQ ID NO: 1 - DNA, 22 bp, Artificial Sequence
  Synthetic oligonucleotide
cgccagggttttcccagtcacg

SEQ ID NO: 2 - DNA, 30 bp, Artificial Sequence
  Synthetic oligonucleotide
gagcggataacaatttcacacaggaaacag

SEQ ID NO: 3 - DNA, 45 bp, Artificial Sequence
  Synthetic oligonucleotide
cccctatttgtttatttttctaaatacattcaatatgtatccgct

SEQ ID NO: 4 - DNA, 32 bp, Artificial Sequence
  Synthetic oligonucleotide
ttaccaatgcttaatcagtgaggcacctatct

SEQ ID NO: 5 - DNA, 31 bp, Artificial Sequence
  Synthetic oligonucleotide
acgtcttgctcgaggccgcgattaaattcca

SEQ ID NO: 6 - DNA, 31 bp, Artificial Sequence
  Synthetic oligonucleotide
aggatggcaagatcctggtatcggtctgcga

SEQ ID NO: 7 - DNA, 28 bp, Artificial Sequence
  Synthetic oligonucleotide
tattgggccctgatcggcacgtaagagg

SEQ ID NO: 8 - DNA, 25 bp, Artificial Sequence
  Synthetic oligonucleotide
caccgggctgcatccgatgcaagtg

SEQ ID NO: 9 - DNA, 29 bp, Artificial Sequence
  Synthetic oligonucleotide
aaagcattctggcgtaacgccgcgttgct

SEQ ID NO: 10 - DNA, 41 bp, Artificial Sequence
  Synthetic oligonucleotide
gtgtcgcttaagttaataacccgctttatcaatcacaaagg

SEQ ID NO: 11 - DNA, 43 bp, Artificial Sequence
  Synthetic oligonucleotide
cctatttgtttatttttctaaatacattcaatatgtatccgct

SEQ ID NO: 12 - DNA, 32 bp, Artificial Sequence; identical to SEQ ID NO: 4
  Synthetic oligonucleotide

SEQ ID NO: 13 - DNA, 24 bp, Artificial Sequence
  Synthetic oligonucleotide
cgccagggttttcccagtcacgac

SEQ ID NO: 14 - DNA, 24 bp, Artificial Sequence
  Synthetic oligonucleotide
agcggataacaatttcacacagga

(19) United States
(12) Patent Application Publication
(10) Pub. No.: US 2005/0064490 A1
(43) Pub. Date: Mar. 24, 2005

(54) Methods of synthesizing polynucleotides using thermostable enzymes

(75) Inventors: Marjorie F. Oleksiak, Raleigh, NC (US); Douglas L. Crawford, Key Biscayne, FL (US)

Correspondence Address: MICHAEL BEST & FRIEDRICH, LLP, ONE SOUTH PINCKNEY STREET, P O BOX 1806, MADISON, WI, 53701, US

(73) Assignee: University of Missouri, Columbia, MO (US)

(21) Appl. No.: 10/947,832

(22) Filed: Sep. 23, 2004

Related U.S. Application Data
(60) Provisional application No. 60/505,300, filed on Sep. 23, 2003.

(51) Int. Cl. C12Q 1/68; C12P 19/34

(52) U.S. Cl. 435/6; 435/91.2

(57) ABSTRACT

Disclosed are methods of synthesizing a polynucleotide complementary to a target polynucleotide include steps of subjecting a non-thermophilic cell comprising a thermostable polymerase to a temperature effective to disrupt the cell to form a reaction mixture, wherein the reaction mixture comprises the target polynucleotide and one or more primers that hybridize to a sequence of the target polynucleotide or to a sequence flanking the polynucleotide and incubating the reaction mixture under conditions whereby the polynucleotide is synthesized. Also disclosed are cell libraries and kits in accordance with the invention.

### CROSS-REFERENCE TO RELATED APPLICATIONS

This application claims benefit of priority under 35 USC § 119 to U.S. Provisional Application Ser. No. 60/505,300, incorporated herein by reference in its entirety.

### STATEMENT REGARDING FEDERALLY SPONSORED RESEARCH OR DEVELOPMENT

The United States government owns rights in the application pursuant to National Science Foundation grant numbers IBN9986602 and DBI-007452.

### INTRODUCTION

The invention relates generally to the fields of molecular biology and biotechnology. In particular, the invention provides methods, cells and kits useful in synthesizing copies of target sequences using thermostable enzymes produced in cyto.

The field of biotechnology conventionally relies on the isolation and purification of enzymes to perform cloning, sequencing, amplification and many other procedures. The discovery of thermostable enzymes has significantly improved these procedures because these enzymes provide higher stability, activity and specificity, attributes that greatly enhance their utility in the laboratory. Current techniques using thermostable enzymes require prior isolation and purification. In conventional use, purified thermostable enzymes are added to an appropriate reaction mixture containing isolated target polynucleotides and are subsequently used at elevated temperatures in the reaction solution. Numerous examples of such techniques exist in the literature, including PCR, sequencing and restriction endonuclease digestion of polynucleotides.

Some techniques have been developed that do not require isolation and purification of target polynucleotides prior to the addition of enzyme. Examples include rolling circle amplification (RCA) using phi 29 DNA polymerase and colony or “dirty” PCR. The starting material for these techniques is typically bacterial cells containing a target plasmid. Instead of isolating the target plasmid from the cellular contaminants these techniques are performed directly on lysed cell preparations.

Although methods exist whereby target polynucleotides may be manipulated without prior isolation and purification, current techniques consistently require the use of purified thermostable enzymes. The necessity of prior enzyme purification substantially increases the costs and processing steps required to utilize molecular techniques.

### BRIEF SUMMARY OF THE INVENTION

In one aspect, the present invention provides methods of synthesizing a polynucleotide complementary to a target polynucleotide. The method includes steps of subjecting a non-thermophilic cell comprising a thermostable polymerase to a temperature effective to disrupt the cell to form a reaction mixture, wherein the reaction mixture comprises the target polynucleotide and one or more primers that hybridize to a sequence of the target polynucleotide or to a sequence flanking the target polynucleotide, and incubating the reaction mixture under conditions whereby a polynucleotide complementary to at least a portion of the target polynucleotide is synthesized.

In another aspect, the invention provides a library comprising a population of non-thermophilic cells comprising a plurality of target polynucleotides, at least one cell in the population comprising a polynucleotide encoding a thermostable polymerase.

The invention further provides a kit for synthesizing a polynucleotide. Kits include a population of non-thermophilic cells, at least one of which comprises a polynucleotide encoding a thermal stable polymerase.

### BRIEF DESCRIPTION OF THE DRAWINGS

FIG. 1 depicts agarose gel verification of amplification of a target 1.1 Kb cDNA in E. coli maintaining a separate plasmid encoding Thermus aquaticus thermostable polymerase.

FIG. 2 depicts agarose gel verification of amplification of a target 1.1 Kb cDNA in E. coli maintaining a separate low copy plasmid encoding Thermus aquaticus thermostable polymerase.

FIG. 3 depicts agarose gel verification of amplification of a plasmid-encoded target 1.1 Kb cDNA in E. coli having chromosomal integrated Thermus aquaticus thermostable polymerase.

FIG. 4 depicts agarose gel verification of amplification of target cDNAs from a library. Library host cells are E. coli expressing Thermus aquaticus thermostable polymerase and maintaining cDNAs on individual plasmids.

FIG. 5 depicts gel verification of amplification of target resistance sequences from high copy, low copy and single copy cloning vectors.

FIG. 6 depicts gel verification of amplification of genomic RNAse I from E. coli.

FIG. 7 depicts gel verification of amplification of target polynucleotides from an uncharacterized genomic library.

### DETAILED DESCRIPTION OF THE INVENTION

Conventional use of enzymes in biotechnology requires purification and storage of purified enzymes, which are then later used in various molecular techniques. As with all purified enzymes, thermostable enzymes typically require separate storage and use conditions. For example, thermostable polymerases are relatively unstable in the buffers in which they are functional. Moreover, stored purified enzymes tend to lose activity over time. The present invention decreases costs of molecular techniques using thermostable enzymes by eliminating the need for separate storage and use of purified enzymes. Moreover, due to a reduction in process steps, the invention achieves ease of use and increased throughput.

In one aspect, the invention provides methods for synthesizing a polynucleotide complementary to a target polynucleotide of interest using a thermostable polymerase. Unlike conventional uses of thermostable polymerases, the methods of the invention do not require prior purification of, e.g., native or recombinant thermostable polymerases. In general, non-thermophilic cells expressing a thermostable polymerase are exposed to a temperature effective to disrupt the cells, thereby exposing the thermostable polymerase to a reaction mixture containing the target polynucleotide and one or more primers under conditions whereby a polynucleotide complementary to at least a portion of the target polynucleotide is synthesized.

Non-thermophilic cells are used as host cells in accordance with the invention. Appropriate non-thermophilic cells are those capable of maintaining polynucleotides encoding thermostable enzymes and expressing functional thermostable enzymes, typically under normal cell culture conditions. At elevated temperatures, non-thermophilic cells are disrupted while the thermostable polymerase retains activity. It will be appreciated that disruption of non-thermophilic cells may include at least heat denaturation and/or destruction of structural and other cell proteins such that the thermophilic polymerase becomes available for use in the reaction mixture. The temperature effective to disrupt the cells, of course, will depend on the type of cell selected as the non-thermophilic host. Suitable non-thermophilic cells include prokaryotic cells and eukaryotic cells. One suitable prokaryotic cell is E. coli, however, any bacterial cell may be selected for use in the methods of the invention by the ordinarily skilled artisan. Suitable eukaryotic cells which may be used include mammalian cells and yeast cells.

Thermostable polymerases expressed by the non-thermophilic cells in accordance with the invention may include, for example, DNA polymerases, RNA polymerases and reverse transcriptases. Selection of the appropriate polymerase will depend on the desired function. Any polymerase may be suitable for use in the present invention as long as it is stable at temperatures that effectively disrupt the host cell proteins. Typically, a thermostable polymerase is one that maintains activity, i.e., is capable of primer extension, at elevated temperatures. Suitable polymerases include those originally isolated from thermophilic bacteria including, but not limited to, Thermococcus litoralis, Bacillus stearothermophilus, Pyrococcus furiosus, Pyrococcus woesei, Thermus aquaticus, Thermus filiformis, Thermus flavus, Thermus thermophilus or Thermotoga maritem. As will be appreciated by the skilled artisan, recombinant polymerases having mutations may also be expressed in the non-thermophilic host cell. The host cell may express more than one polymerase.

The polynucleotides encoding the thermostable polymerase may be encoded on a cloning vector and introduced into the host cell by standard methods. The vector may be an autonomously replicating polynucleotide, such as a plasmid, that is maintained in the host cell cytoplasm, or may be integrated into the genome of the host cell. Representative examples of cloning vectors which may be used include viral particles, baculovirus, phage, plasmids, phagemids, cosmids, phosmids, bacterial artificial chromosomes, viral DNA (e.g. vaccinia, adenovirus, foul pox virus, pseudorabies and derivatives of SV40), P1-based artificial chromosomes, yeast plasmids and yeast artificial chromosomes. As will be appreciated by those of skill in the art, any cloning vector may be used as long as it is replicable and viable in the host. Moreover, methods of integrating a polynucleotide encoding a thermostable polymerase into the genome of a host cell are known and may include use of transposable genetic elements, viral vectors, or allelic exchange using recombination enzymes.

Polynucleotides encoding the thermostable polymerase present in the non-thermophilic cell may be operably connected to promoters functional in the host cell. Promoters may be constitutive or inducible. As will be recognized by the skilled artisan, suitable promoters useful in bacteria include lacI, lacZ, T3, T7, gpt, lambda P_{R}, P_{L} and trp. Suitable promoters useful in eukaryotic cells include CMV immediate early, HSV thymidine kinase, early and late SV40, LTRs from retrovirus, and mouse metallothionein-I. Additional control sequences, e.g., enhancers, may also be operably connected to the promoter or coding sequence. Selection of the appropriate promoter and/or control sequences is well within the level of ordinary skill in the art.

Polynucleotides encoding the thermostable polymerase may also include one or more sequences encoding selectable markers to provide a phenotypic trait for selection of transformed host cells. Examples of selectable markers, use of which are standard in the art, include antibiotic resistance, such as, e.g., tetracycline or ampicillin resistance in E. coli.

The target polynucleotide may be introduced into the non-thermophilic cell using standard methods, such as for example, transformation or transfection of the cell with a cloning vector, or may be added to the reaction mixture as isolated DNA. Alternatively, a second host cell comprising the target polynucleotide may be co-cultured with the cell transformed with the polynucleotide encoding the thermostable polymerase or added to the reaction mixture. When present in the reaction mixture as isolated DNA, the target polynucleotide may be linear or episomal. As will be appreciated by those of skill in the art, embodiments of the invention wherein the target polynucleotide is introduced directly into the non-thermophilic cell eliminate the need for isolation and purification of the target prior to polymerization. In these embodiments, the target polynucleotide may be encoded on the vector that also encodes the thermostable polymerase, or may be introduced on a second cloning vector, or may be integrated into the non-thermophilic cell genome. The need for isolation and purification of the target polynucleotide prior to polymerization is also avoided in embodiments wherein the target is encoded within a second cell that is co-cultured with the non-thermophilic cell comprising the thermostable polymerase.

The reaction mixture is formed upon disruption of the non-thermophilic cell. Disruption of the cell and denaturation of cellular proteins provides for the specific polymerization of the complement to the target polynucleotide via primer hybridization and extension by the thermophilic polymerase. Primers may hybridize either to a sequence of the target polynucleotide, or to a sequence flanking the target polynucleotide, for example, a sequence of a cloning vector such as a multicloning sequence. Primers that also hybridize to the complement of the target polynucleotide are optionally included in the reaction mixture to provide for second strand synthesis. In addition to primers that hybridize to the target polynucleotide (or a sequence flanking the target) and its complement, the reaction mixture suitably further contains buffers that may be optimized by the skilled artisan to adjust the stringency of the hybridization conditions and/or optimize performance of the polymerase. Nucleotides, such as deoxyribonucleotides or ribonucleotides, are also suitably included in the reaction mixture, and may be modified to incorporate labels, such as radioactivity, fluorescence molecules or biotin, useful in downstream applications. Labeled dideoxynucleotides may be included for sequencing applications.

It will be understood that the non-thermophilic cell may be added to a solution containing primers, nucleotides and buffer prior to disrupting the cell to form the reaction mixture, or alternatively, the solution containing primers, nucleotides and buffer may be added to the disrupted cell to form the reaction mixture. It will further be appreciated that the cell, target polynucleotide, buffer, primers and nucleotides may be added to the reaction mixture in any order and that the temperature effective to disrupt the cell may be applied at any time without departing from the invention.

Polymerization of the polynucleotide complementary to the target suitably may be accomplished via thermocycling, or PCR. Thermocycling conditions may be empirically determined by the skilled artisan without undue experimentation, taking into consideration factors such the identity of the polymerase, length and base composition of the primers, as well as the ionic strength of the reaction buffer. A representative example of a suitable cycling scheme is as follows: 32 cycles of 94° C. for 20 seconds to denature DNA, followed by 70° for 1-4 minutes to provide for primer annealing and extension and, following these cycles, a final extension of 72° C. for 15 minutes. Isothermal polymerization is also encompassed by the present invention. In isothermal polymerization, DNA helicases are used to separate the target strands, instead of, for example, heat. DNA helicases may also be used in conjunction with thermocycling.

The invention also provides cell libraries. Cell libraries in accordance with the invention include, but are not limited to, cDNA libraries, genomic libraries or expression libraries. Thermostable polymerase is expressed in at least one cell of the library permitting polymerization of polynucleotides complementary to genomic or cDNA inserts upon disruption of the cells under the appropriate conditions, as described above.

Kits including non-thermophilic cell populations, at least one cell of which expresses a thermostable polymerase are also encompassed by the present invention. In some embodiments, the kits further include a polynucleotide comprising a cloning vector Suitably, the cells are competent cells. Optional further components in the kit include primers that hybridize to the cloning vector, at least one reaction buffer, nucleotides, at least one restriction endonuclease, a DNA helicase and instructions for use of the kit according to the method described herein. Suitably, the cloning vector includes a multiple cloning sequence. The nucleotides may be labeled.

As will be apparent to those of skill in the art from the foregoing description and following examples, the present invention may be adapted to include use of any thermostable enzyme that can be expressed in a non-thermophilic cell, including, but not limited to, polymerases, ligases, restriction endonucleases, DNA helicases and methylases. Moreover, the invention may be adapted to make use of enzymes functional under other extreme conditions, i.e., those isolated from, e.g., halophiles, etc.

The following examples are provided to assist in a further understanding of the invention. The particular materials and conditions employed are intended to be further illustrative of the invention and are not limiting upon the reasonable scope thereof.

### EXAMPLES

### Example 1

### Plasmid-Encoded Thermal Stable Polymerase

Thermus aquaticus (Taq) DNA polymerase was cloned in pUC18, and co-transfected into host E. coli cells with a separate plasmid encoding a 1.1 Kb J5 target cDNA. Taq expression was induced with 0.1, 0.5, 1, and 5 mM IPTG.

One to four microliters of overnight host cell bacterial culture was added to a solution containing 10 pmoles each of forward and reverse pUC-derived plasmid-specific primers:

| | |
|---|---|
| 5′CGCCAGGGTTTTCCCAGTCACG3′ | [SEQ ID NO.: 1] |
| 5′GAGCGGATAACAATTTCACACAGGAAACAG3′ | [SEQ ID NO.: 2] |

The reaction solution also contained 0.2 mM dNTPs and reaction buffer with detergents and DMSO at the following final concentrations: 50 mM Tris HCl, pH 9.2 (25° C.), 16 mM (NH₄)2SO₄, 2.25 mM MgCl₂, 2% (v/v) DMSO, 0.1% (v/v) Tween 20.

The mixture of bacteria and reaction solution was heated to 80° C. for 20 seconds to denature bacterial proteins, but not the Taq DNA polymerase, and subjected to thermal cycling as follows: 32 cycles of 94° C. for 20 seconds (DNA denaturation) followed by 70° C. for 1-4 minutes (primer annealing and extension; time was dependent on target size) and a final extension of 72° C. for 15 minutes. To screen for polymerase activity, 5-10% of the final volume was resolved on a 1% agarose gel.

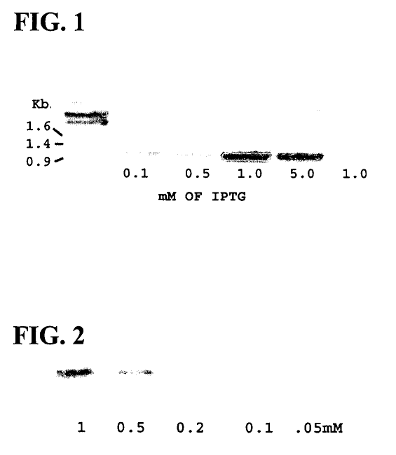

The results are shown in FIG. 1. Amplification of 1.1 Kb J5 cDNA, seen in lanes 2-5, is affected by IPTG induction of the thermostable polymerase. Lane 6 is a control lacking thermostable polymerase.

### Example 2

### Low Copy Plasmid-Encoded Thermostable Polymerase

The Taq coding sequence was excised from pUC18 with AflIII and XbaI, blunt-ended with T4 DNA polymerase, gel purified, and ligated into the blunted HindIII site of a low copy plasmid, pACYC184, having a p15A origin of replication. The plasmid was transfected into bacteria (E. coli JM109 and DH5α) also containing J5 cDNA in pBS SK-(Stratagene). Screening for polymerase activity was conducted as described in Example 1.

The results are shown in FIG. 2. Lanes 1-5 show decreasing concentrations of IPTG. Again, amplification of target J5 cDNA was affected by IPTG induction of thermostable polymerase.

### Example 3

### High, Low and Single Copy Expression Vectors Encoding Thermostable Polymerase and Amplification of Resistance Sequences on the Expression Vectors

A. Construction of Expression Vectors

A sequence encoding Taq thermostable polymerase was amplified and cloned into the EcoRI site of pUC18 (ampicillin resistant) under the control of the lacZ promoter. This plasmid was digested using PvuI and TfiI. After digestion, plasmid fragments were blunt-ended with T4 DNA polymerase and purified on a low melting agarose gel. The fragment encoding Taq under the control of the lacZ promoter was ligated in low copy (approximately 40 copies/cell) plasmid pSMART LCKan (kanamycin resistant, Lucigen Corp, Middleton Wis.) and single copy plasmid pSMART VC (chloramphenicol resistant, Lucigen Corp, Middleton Wis.) using T4 DNA ligase in a 10 μl volume containing 25 ng vector DNA and 50 ng insert DNA. Electrocompetent E. coli cells 10 G (Lucigen Corp, Middleton Wis.) were transformed with the ligase mixture. Transformed cells were grown on TB medium for 1 hour. Kanamycin resistant (pSMART LCKan) or chloramphenicol resistant (pSMART VC) colonies arising from the transformation were selected on the appropriate antibiotic plate. The clones which contained the lacZ/Taq DNA polymerase insert were selected by size analysis on agarose electrophoresis gels.

B. PCR Amplification of Resistance Sequences on Expression Vectors

Single colonies of bacteria containing recombinant pUC19/Taq, pSMART LCKan/Taq and pSMART VC/Taq were picked and each were resuspended in 100 μl of lysis buffer (10 mM Tris-HCl pH 7.5, 1 mM EDTA, 1 mM DTT, 50% Glycerol, 0.1% Triton X-100, 10 μg RNase A, 1 unit bacteriophage T4 lysozyme). The lysis extract was incubated at room temperature for 10 minutes, at 70° C. for 10 minutes and on ice for 5 minutes. The lysis extract was spun at 13,000 RPM for 5 minutes.

Supernatants from the lysis extracts were then subjected to PCR to amplify the resistance sequences from each of pUC19/Taq, pSMART LCKan/Taq and pSMART VC/Taq. PCR reactions consisted of 5 μl of lysis cell extract mixed with a reaction buffer (10 mM Tris-HCl pH 9.0, 50 mM potassium chloride, 1.5 mM magnesium chloride, 0.1% Triton X-100, 25 μM each of the deoxyribonucleotide triphosphates (dGTP, dCTP, dTTP, and dATP) and primer pairs as indicated below in a 50 μl final volume.

Resistance

| | | |
|---|---|---|
| Vector | sequence | Primer sequences |

| | | |
|---|---|---|
| pUC19/Taq | Ampicillin | Forward: |
| (high copy) | | 5′-CCCCTATTTGTTTATTTTT |
| | | CTAAATACATTCAATATGTATC |
| | | CGCT-3′ |
| | | [SEQ ID NO.: 3] |
| | | Reverse: |
| | | 5′-TTACCAATGCTTAATCAGT |
| | | GAGGCACCTATCT-3′ |
| | | [SEQ ID NO.: 4] |
| pSMART | Kanamycin | Forward: |
| LCKan/Taq | | 5′-ACGTCTTGCTCGAGGCCGC |
| (low copy) | | GATTAAATTCCA-3′ |
| | | [SEQ ID NO.: 5] |
| | | Reverse: |
| | | 5′-AGGATGGCAAGATCCTGGT |
| | | ATCGGTCTGCGA-3′ |
| | | [SEQ ID NO.: 6] |
| pSMART | Chloramphenicol | Forward: |
| VC/Taq | | 5′-TATTGGGCCCTGATCGGCA |
| (single | | CGTAAGAGG-3′ |
| copy) | | [SEQ ID NO.: 7] |
| | | Reverse: |
| | | 5′-CACCGGGCTGCATCCGATG |
| | | CAAGTG-3′ |
| | | [SEQ ID NO.: 8] |

Thermal cycling was conducted as follows: 25 cycles of 94° C. for 15 seconds, followed by 60° C. for 15 seconds, followed by 72° C. for 1 minute and a final extension of 72° C. for 10 minutes.

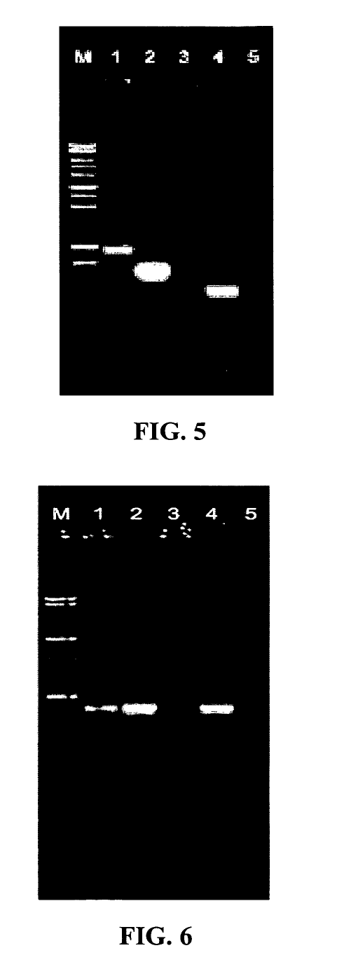

Completed PCR reactions were subjected to gel electrophoresis. The results are shown in FIG. 5. Lane M contains a standard 1 Kb Ladder. Lane 1 shows amplification of the ampicillin resistance sequence from a high copy vector from a single bacterial colony. Lane 2 shows amplification of kanamycin resistance sequence from a low copy vector from a single bacterial colony. Lane 3 contains low copy vector with no Taq insert from a single bacterial colony. Lane 4 shows amplification of chloramphenicol resistance sequence from a single copy vector from a single bacterial colony. Lane 5 contains single copy vector with no Taq insert from a single bacterial colony. These results indicate that the invention is able to amplify DNA fragments from a single bacterial colony without prior purification of the DNA template or DNA polymerase.

### Example 4

### Chromosomal Integration of Thermostable Polymerase

Plasmid pAG408 was digested with KpnI to delete coding sequences for green fluorescent protein and 3′-aminoglycoside phosphotransferase and re-ligated to restore the original plasmid, pBSL202. The Taq coding sequence was cut out of pUC18 with AflIII and blunted using T4 DNA polymerase followed by XbaI digestion. Gel purified Taq coding sequence with a blunt end and an XbaI overhang was ligated into the blunted NotI and sticky XbaI site of pBSL202 (pAG408 derived), forming a mini-Tn5transposon derivative for the delivery of Taq into gram-negative bacteria. This is a suicide mini-transposon delivery plasmid that is R6K based and unable to survive in recipients lacking the Pir protein. The plasmid was propogated in E. coli S17-1 (lambda pir), and electrocompetent JM109 cells previously transfected with a plasmid encoding J5 cDNA were tranfected with the purified plasmid encoding Taq and selected with gentamycin (30 ug/ml). Gentamycin resistant colonies were screened for polymerase activity as described in Example 1.

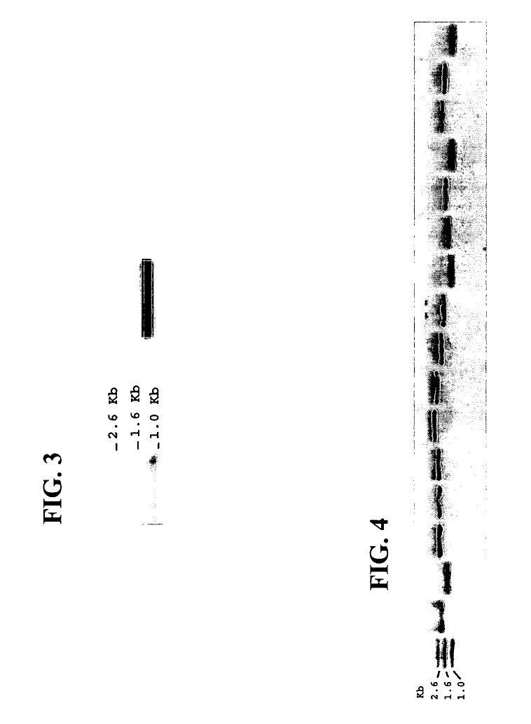

The results are shown in FIG. 3. Lane 3 contains a 1.1 Kb band of amplified J5 cDNA.

### Example 5

### Amplification of Target Polynucleotides from a cDNA Library

Amplification of sixteen different cDNA products was conducted using the methods described in Example 1. Bacteria expressing thermostable polymerase were transfected with target cDNAs reverse transcribed from mRNAs derived from a heart library. Expression of polymerase was induced with 0.5 mM IPTG.

FIG. 4 depicts amplification of sixteen target cDNAs. The size variation represents different sized cDNAs (4,000-1,200 bp).

### Example 6

### Amplification of Target Sequence (RNase I gene) from E coli Genomic DNA Using High, Low and Single Copy Taq DNA Polymerase Expression Vectors

Single colonies of E. coli having chromosomal RNAse I gene as the target DNA, and containing recombinant Taq cloned on a high, low or single copy vector, were picked and resuspended in 100 μl of lysis buffer (10 mM Tris-HCl pH 7.5, 1 mM EDTA, 1 mM DTT, 50% Glycerol, 0.1% Triton X-100, 10 μg RNase A, 1 unit bacteriophage T4 lysozyme). The lysis extract was incubated at room temperature for 10 minutes, at 70° C. for 10 minutes and on ice for 5 minutes, followed by centrifugation at 13,000 RPM for 5 minutes.

Supernatants from the lysis extracts were then subjected to PCR. Reactions consisted of 5 μl of supernatant mixed with a reaction buffer (10 mM Tris-HCl pH 9.0, 50 mM potassium chloride, 1.5 mM magnesium chloride, 0.1% Triton X-100, 25 uM each of the deoxyribonucleotide triphosphates (dGTP, dCTP, dTTP, and dATP) and primer pairs directed to the RNAse I gene as indicated below in a 50 μl final volume.

| | |
|---|---|
| Forward: | |
| 5′-AAAGCATTCTGGCGTAACGCCGCGTTGCT- | [SEQ ID NO.: 9] |
| 3′ | |
| Reverse: | |
| 5′-GTGTCGCTTAAGTTAATAACCCGCTTTATCA | [SEQ ID NO.: 10] |
| ATCACAAAGG-3′ | |

Thermal cycling was conducted as follows: 25 cycles of 94° C. for 15 seconds, followed by 60° C. for 15 seconds, followed by 72° C. for 1 minute and a final extension of 72° C. for 10 minutes.

Completed PCR reactions were subjected to gel electrophoresis. The results are shown in FIG. 6. Lane M contains a standard 1 Kb Ladder. Lane 1 shows amplification of the E. coli RNase I gene with RNase I forward and reverse primers by Taq Polymerase in high copy plasmid from a single bacterial colony. Lane 2 shows amplification of the E. coli RNase I gene with RNase I forward and reverse primers by Taq Polymerase in low copy plasmid from a single bacterial colony. Lane 3 shows no E. coli RNase I gene DNA amplification with RNase I forward and reverse primers from a single bacterial colony containing low copy vector with no insert. Lane 4 shows amplification of the E. coli RNase I gene with RNase I forward and reverse primers by Taq Polymerase in single copy plasmid from a single bacterial colony. Lane 5 shows no E. coli RNase I gene DNA amplification with RNase I forward and reverse primers from a single bacterial colony containing a single copy vector with no insert.

The results indicate that the invention is able to amplify DNA fragments from a single bacterial colony without prior purification of the DNA template or DNA polymerase. The amplification product in lane 4 was gel purified and the nucleotide sequence was determined using fluorescent dye chemistry (Applied Biosystems, Foster City, Calif.) to be that expected from the RNase I gene product.

### Example 7

### Amplification of Target Polynucleotides from a Genomic Library

A recombinant library (Obsidian Library Y4.12MC) of genomic DNA prepared from an uncharacterized thermophilic strain of bacteria was constructed in pSMART HCKan using standard methods. The recombinant library was transformed into electrocompetent E. coli 10 G cells previously transformed with single copy vector pSMART VC containing Taq, as described in Example 3. A single colony of bacteria containing pSMART VC/Taq and pSMART HCKan/random DNA insert was picked and resuspended in 100 μl of lysis buffer (10 mM Tris-HCl pH 7.5, 1 mM EDTA, 1 mM DTT, 50% Glycerol, 0.1% Triton X-100, 10 μg RNase A, 1 unit bacteriophage T4 lysozyme). The lysis extract was incubated at room temperature for 10 minutes, at 70° C. for 10 minutes and on ice for 5 minutes, followed by centrifugation at 13,000 RPM for 5 minutes.

Supernatants from the lysis extracts were then subjected to PCR. PCR reactions consisted of 5 μl of supernatant mixed with a reaction buffer (10 mM Tris-HCl pH 9.0, 50 mM potassium chloride, 1.5 mM magnesium chloride, 0.1% Triton X-100, 25 uM each of the deoxyribonucleotide triphosphates (dGTP, dCTP, dTTP, and dATP) and primer pairs as indicated below in a 50 μl final volume.

Ampicillin forward:

| | |
|---|---|
| 5′-CCTATTTGTTTATTTTTCTAAATACATTCAA | [SEQ ID NO.: 11] |
| TATGTATCCGCT-3′ | |
| Ampicillin reverse: | |
| 5′-TTACCAATGCTTAATCAGTGAGGCACCTATC | [SEQ ID NO.: 12] |
| T-3′ | |
| Z-forward: | |
| 5′-CGCCAGGGTTTTCCCAGTCACGAC-3′ | [SEQ ID NO.: 13] |
| Z-reverse: | |
| 5′-AGCGGATAACAATTTCACACAGGA-3′ | [SEQ ID NO.: 14] |

Thermal cycling was conducted as follows: 25 cycles of 94° C. for 15 seconds, followed by 60° C. for 15 seconds, followed by 72° C. for 1 minute and a final extension of 72° C. for 10 minutes.

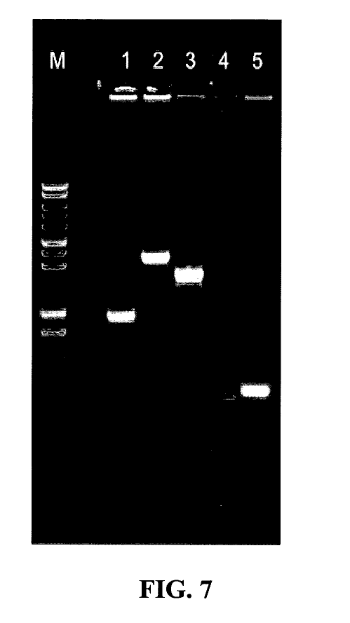

Completed PCR reactions were subjected to gel electrophoresis. The results are shown in FIG. 7. Lane M contains a standard 1 Kb Ladder. Lane 1 shows PCR amplification of pUC 18 sequences by Taq polymerase cloned in single copy from a single bacterial colony using Amp primers. Lanes 2, 3, 4 and 5 show PCR amplification of plasmid DNA from Obsidian Library Y4.12MC by Taq polymerase in single copy from a single bacterial colony using Z-forward and Z-reverse primers.

### Example 8

### Thermostable Restriction Enzyme

A thermostable restriction enzyme is used to screen plasmid-propagated target polynucleotides. A high copy plasmid is used to clone PCR products and clones are transfected into E. coli having a chromosomally integrated coding sequence for a restriction enzyme under the control of a constituitive promoter. The multiple cloning site of the high copy plasmid is flanked by recognition sites for the thermostable restriction enzyme. In order to test for presence or absence of the inserted PCR products, an aliquot of the cells, e.g., 10 μl of an overnight culture, is heated to 80° C. for 20 seconds in the presence of 10 μl of 2×buffer optimized for the activity of the restriction enzyme. At this temperature, denaturation of bacterial proteins but not the thermostable restriction enzyme occurs. The heated bacterial/buffer solution is then incubated at the appropriate temperature for optimal activity of the thermostable restriction enzyme until restriction occurs. The digested product is resolved using gel electrophoresis. Due to the high copy number of the plasmid, signal (restriction enzyme digest) from the plasmid will overwhelm background noise from bacterial genomic DNA also cut with the theromostable restriction enzyme.

### Example 9

### Thermostable Reverse Transcriptase

A thermostable reverse transcriptase integrated in chromosomal DNA of bacteria is used to reverse transcribe cDNAs from RNAs produced in mammalian cells. These cDNAs represent a linear amplification of target gene and have incorporated label for downstream applications. The target plasmids with a gene of interest operably connected to a promoter are first propagated in the host bacteria. The cells are cultured under conditions allowing for induction of the promoter and the production of RNA from the DNA. The bacteria are then heated to 80° C. for 20 seconds in the presence of buffer optimized for the activity of the reverse transcriptase. At this temperature, the cells are lysed and the bacterial proteins are denatured. Labeled nucleotide triphosphates and target-specific primers are then added to the reaction mixture. The reaction mixture will be cooled to allow the target specific primer to bind to target. Subsequently, the thermostable reverse transcriptase can reverse transcribe specific cDNAs using target specific primer bound to the template RNA. The end result is linear amplification of single-stranded cDNAs that can be used in downstream applications.

### Example 10

### Thermostable Methylase

A thermostable methylase is encoded on plasmid propagated in bacteria and used to methylate a high-copy plasmid encoding a target polynucleotide. Host cells are heated to 80° C. for 20 seconds in the presence of buffer optimized for the methylse activity to denature bacterial proteins but not the thermostable methylase. Methylated high-copy plasmids can be used directly from the reaction mixture. Due to the high copy number of the plasmid, methylated plasmid will overwhelm background noise from bacterial genomic DNA.

### Example 11

### Sequencing

A single colony of bacteria containing recombinant Taq polymerase cloned on a high, low or single copy vector and a target polynucleotide on another plasmid is picked and resuspended in 11 μl of water. One microliter of primer (4 pmol), 2 μl BigDye (Applied Biosystems) and 6 μl 2.5×buffer [5× is 400 mM Tris pH 9, 10 mM MgCl₂] is added. The reaction mix is placed in a thermal cycling instrument with an initial 95° C. for 3 minutes, then 50 cycles of 96° C. for 10 seconds, 58° C. for 4 minutes, and finished with 72° C. for 7 minutes. The reaction is cleaned by ethanol precipitation or spin column chromatography, dried at 70° C. for 15 minutes, and resuspended in 20 μl formamide before loading onto an ABI 310 automated DNA sequencer.

As used in this specification and the appended claims, the singular forms “a,” “an,” and “the” include plural referents unless the content clearly dictates otherwise. Thus, for example, reference to a composition containing “a polynucleotide” includes a mixture of two or more polynucleotides. It should also be noted that the term “or” is generally employed in its sense including “and/or” unless the content clearly dictates otherwise.

All publications, patents and patent applications referenced in this specification are indicative of the level of ordinary skill in the art to which this invention pertains. All publications, patents and patent applications are herein expressly incorporated by reference to the same extent as if each individual publication or patent application was specifically and individually indicated by reference. In case of conflict between the present disclosure and the incorporated patents, publications and references, the present disclosure should control.

The invention has been described with reference to various specific embodiments and techniques. However, it should be understood that many variations and modifications may be made while remaining within the spirit and scope of the invention.

1. A method of synthesizing a polynucleotide complementary to a target polynucleotide comprising:
a) subjecting a non-thermophilic cell comprising a thermostable polymerase to a temperature effective to disrupt the cell to form a reaction mixture, wherein the reaction mixture comprises the target polynucleotide and one or more primers that hybridize to a sequence of the target polynucleotide or to a sequence flanking the target polynucleotide; and
b) incubating the reaction mixture under conditions whereby a polynucleotide complementary to at least a portion of the target polynucleotide is synthesized.

2. The method of claim 1, wherein the cell comprises a polynucleotide encoding the thermostable polymerase operably connected to a promoter functional in the cell.

3. The method of claim 2, wherein the polynucleotide encoding the thermostable polymerase is integrated into the genome of the cell.

4. The method of claim 2, wherein the cell comprises a cloning vector comprising a sequence encoding the thermostable polymerase.

5. The method of claim 4, wherein the cloning vector further comprises the target polynucleotide.

6. The method of claim 1, wherein the cell comprises a cloning vector comprising the target polynucleotide.

7. The method of claim 1, wherein the cell comprises a first cloning vector comprising a sequence encoding the thermostable polymerase, and wherein the cell further comprises a second cloning vector comprising the target polynucleotide.

8. The method of claim 1, wherein the reaction mixture further comprises one or more primers that hybridize to the complementary polynucleotide synthesized in step b).

9. The method of claim 1, wherein the reaction mixture comprises one or more deoxyribonucleotides, ribonucleotides or dideoxynucleotides.

10. The method of claim 1, wherein the reaction mixture comprises a reaction buffer.

11. The method of claim 1, wherein the reaction mixture comprises a cloning vector comprising the target polynucleotide.

12. The method of claim 11, wherein a second non-thermophilic cell comprises the cloning vector.

13. The method of claim 11, wherein a virus comprises the cloning vector.

14. The method of claim 11, wherein the cloning vector is linearized.

15. The method of claim 11, wherein the cloning vector is episomal.

16. The method of claim 1, wherein the thermostable polymerase is a reverse transcriptase, an RNA polymerase or a DNA polymerase.

17. The method of claim 1, wherein the thermostable polymerase is selected from polymerases natively expressed in Thermococcus litoralis, Bacillus stearothermophilus, Pyrococcus furiosus, Pyrococcus woesei, Thermus aquaticus, Thermus filiformis, Thermus flavus, Thermus thermophilus or Thermotoga maritema or recombinant variants thereof.

18. The method of claim 1, wherein the conditions of step b) include thermal cycling.

19. The method of claim 1, wherein the reaction mixture further comprises a DNA helicase.

20. The method of claim 1, wherein the cell is a prokaryotic cell.

21. The method of claim 20, wherein the prokaryotic cell is E. coli.

22. The method of claim 1, wherein the cell is a eukaryotic cell.

23. The method of claim 22, wherein the cell is a mammalian cell.

24. The method of claim 1, wherein the cell is a yeast cell.

25. A library comprising a population of non-thermophilic cells comprising a plurality of target polynucleotides, at least one cell in the population comprising a polynucleotide encoding a thermostable polymerase.

26. A kit for synthesizing a polynucleotide according to the method of claim 1 comprising: a population of non-thermophilic cells, at least one cell in the population comprising a polynucleotide encoding a thermal stable polymerase.

27. The kit of claim 26, further comprising a polynucleotide comprising a cloning vector.

28. The kit of claim 26, wherein the cells are competent cells.

29. The kit of claim 27, further comprising one or more primers that hybridize to the cloning vector.

30. The kit of claim 27, wherein the cloning vector comprises a multi-cloning sequence.

31. The kit of claim 26, further comprising at least one reaction buffer.

32. The kit of claim 26, further comprising one or more deoxyribonucleotides, ribonucleotides or dideoxynucleotides.

33. The kit of claim 32, wherein at least one of the deoxyribonucleotides, ribonucleotides or dideoxynucleotides comprises a detectable label.

34. The kit of claim 26, further comprising at least one restriction endonuclease.

35. The kit of claim 26, further comprising a ligase.

36. The kit of claim 26, further comprising a DNA helicase.

37. The kit of claim 26, further comprising instructions for use of the kit.
